(19) United States
(12) Patent Application Publication
(10) Pub. No.: US 2024/0169537 A1
(43) Pub. Date: May 23, 2024

(54) METHODS AND SYSTEMS FOR AUTOMATIC CT IMAGE QUALITY ASSESSMENT

(71) Applicant: GE Precision Healthcare LLC, Waukesha, WI (US)

(72) Inventors: Mathieu Bedez, Strasbourg (FR); Jean Baptiste Wahl, Strasbourg (FR); Estelle Spasic, Strasbourg (FR)

(21) Appl. No.: 18/485,134

(22) Filed: Oct. 11, 2023

Related U.S. Application Data
(60) Provisional application No. 63/384,383, filed on Nov. 18, 2022.

(51) Int. Cl. G06T 7/00; G16H 30/20; G16H 40/63

(52) U.S. Cl. CPC G06T 7/0014; G16H 30/20; G16H 40/63; G06T 2200/04; G06T 2200/24; G06T 2207/10081; G06T 2207/20081; G06T 2207/20104; G06T 2207/30168

(57) ABSTRACT

Methods and systems are provided for automatically generating an image quality score for a computed tomography (CT) image within an image quality assessment system. In one example, a method for an image quality assessment system comprises receiving a selection of a medical image from a user of the image quality assessment system; generating an image quality score for the selected medical image, the image quality score generated using a trained machine learning (ML) model; displaying the selected medical image and the image quality score in a graphical user interface (GUI) on a display device of the image quality assessment system; receiving an adjusted image quality score of the medical image from the user via the GUI; and using the adjusted image quality score to retrain the ML model.

### CROSS REFERENCE TO RELATED APPLICATIONS

The present application claims priority to U.S. Provisional Application No. 63/384,383, entitled “METHODS AND SYSTEMS FOR AUTOMATIC CT IMAGE QUALITY ASSESSMENT,” and filed on Nov. 18, 2022. The entire contents of the above-listed application are hereby incorporated by reference for all purposes.

### TECHNICAL FIELD

Embodiments of the subject matter disclosed herein relate to medical imaging, and in particular, assessing a quality of computed tomography images.

### BACKGROUND

In a computed tomography (CT) imaging system, an electron beam generated by a cathode is directed towards a target within an X-ray tube. A fan-shaped or cone-shaped beam of X-rays produced by electrons colliding with the target is directed towards an object, such as a patient. After being attenuated by the object, the X-rays impinge upon an array of radiation detectors, generating an image.

A quality of the image may be relied on for patient diagnostics. The quality of the image may be assessed in terms of spatial resolution, contrast, noise, a presence and/or number of artifacts generated, and/or other characteristics. The quality of the image may depend on various acquisition parameters of the CT imaging system configured by a user of the CT imaging system, such as radiation dosage (e.g., an amount of current applied to the cathode), focal spot size and/or shape; sampling frequency; a selected field of view; slice thickness, a selected reconstruction algorithm; and others. As a result, image quality may not be consistent across users and/or scans performed, where some parameter configurations may generate higher quality images than other parameter configurations. However, feedback regarding how the CT imaging system could be differently configured to increase the quality of reconstructed images may not be available, and learning how to configure the settings to consistently generate high quality images may entail years of user experience via a trial and error process.

As relevant technologies evolve, users increasingly request and expect higher levels of automation from radiology applications, where artificial intelligence (AI) techniques are used to automate aspects of a CT system and aid users in obtaining desired results.

### SUMMARY

The current disclosure at least partially addresses one or more of the above identified issues by a method for an image quality assessment system, the method comprising receiving a selection of a medical image from a user of the image quality assessment system; generating an image quality score for the selected medical image, the image quality score generated using a trained machine learning (ML) model; displaying the selected medical image and the image quality score in a graphical user interface (GUI) on a display device of the image quality assessment system; receiving an adjusted image quality score of the medical image from the user via the GUI; and using the adjusted image quality score to retrain the ML model.

It should be understood that the summary above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

Various aspects of this disclosure may be better understood upon reading the following detailed description and upon reference to the drawings in which:

FIG. 1 is a schematic block diagram of an image quality assessment system, in accordance with one or more embodiments of the present disclosure;

FIG. 2 is a schematic block diagram indicating a general strategy for generating a image quality score of a medical image, in accordance with one or more embodiments of the present disclosure;

FIG. 3 is a schematic block diagram showing an overview of steps of a procedure for generating the image quality score, in accordance with one or more embodiments of the present disclosure;

FIG. 4 is a flowchart illustrating an exemplary method for generating and displaying the image quality score, in accordance with one or more embodiments of the present disclosure;

FIG. 5 is a flowchart illustrating an exemplary method for ingesting and processing one or more Digital Imaging and Communications in Medicine (DICOM) files, in accordance with one or more embodiments of the present disclosure;

FIG. 6 is a flowchart illustrating an exemplary method for displaying image quality scores on a display device and receiving user feedback, in accordance with one or more embodiments of the present disclosure;

FIG. 7 is an exemplary image quality GUI of the image quality assessment system, in accordance with one or more embodiments of the present disclosure;

FIG. 8A is a first example of a display panel indicating low-level image quality metrics displayed in an image quality GUI of the image quality assessment system, in accordance with one or more embodiments of the present disclosure;

FIG. 8B is a second example of a display panel indicating low-level image quality metrics displayed in an image quality GUI of the image quality assessment system, in accordance with one or more embodiments of the present disclosure;

FIG. 9 is an example of a display panel indicating low-level image quality metrics for a plurality of slices of an image, displayed in the GUI of the image quality assessment system, in accordance with one or more embodiments of the present disclosure; and

FIG. 10 is an example of a display panel showing a slice of a medical image displayed in the GUI of the image quality assessment system, in accordance with one or more embodiments of the present disclosure.

### DETAILED DESCRIPTION

The methods and systems described herein relate to automatically generating an assessment of a quality of a medical image acquired via a medical imaging system, such as a computed tomography (CT) image acquired via a CT system. While descriptions of the systems and methods herein may refer to CT images and systems, it should be appreciated that the systems and methods may also apply to other types of medical images. The assessment may be used to optimize acquisition parameters of the medical imaging system for various imaging tasks and scenarios. For example, image quality assessments may be used to train one or more machine learning (ML) models to predict a set of acquisition parameters for an imaging task that maximizes the quality of images reconstructed using the set of acquisition parameters.

A quality of a CT image may be degraded due to distortions during image acquisition and processing. Examples of distortions include noise, blurring, and/or a presence of artifacts such as ring artifacts, compression artifacts (e.g., graininess of an image), and/or streaking. The quality of a distorted or degraded CT image may be evaluated using various metrics, which may estimate noise and/or contrast levels, spatial resolution, or other aspects of the CT image.

For example, the quality of a CT image can be evaluated with respect to noise. Noise in a CT image is based on photon counting statistics, where a level of noise in the image is inversely related to a number of photons counted at a detector of the CT image (e.g., noise in the image decreases as a number of counted photons increases). Thus, the image noise is dependent on a radiation dosage, where increasing the dosage reduces noise. Noise in a CT image may be measured as a total amount of noise, or a signal-to-noise ratio (SNR). Other factors that affect the level of noise in a CT image are slice thickness, where thicker slices increase SNR; patient size, where larger patients reduce SNR; and a reconstruction algorithm selected to reconstruct the image, where uniform regions of the image may have lower noise than highly structured regions. Noise has various measurable aspects, including magnitude (e.g., the denominator of the SNR), texture (e.g., a quality of the noise, which may be measured by computing a noise power spectrum (NPS)), and uniformity (e.g., a measurement of variations in magnitude or texture across the image).

The quality of a CT image can also be evaluated with respect to contrast. Contrast is the difference between values in different parts of the image, which may allow for distinguishing between different types of tissues. Contrast is typically expressed in terms of a contrast-to-noise ratio (CNR), which is the ratio of the contrast between a signal in a given region and a background. The CNR can be calculated based on measurements within regions of interest (ROI) of the CT image. As the CNR increases, the ROI may be more easily visualized with respect to the background.

The quality of a CT image can also be evaluated with respect to spatial resolution. Spatial resolution refers to the ability to distinguish between objects of different densities in the image. A high spatial resolution may be relied on for discriminating between structures located within a close proximity to each other. Factors that impact spatial resolution include detector size, slice thickness, edge enhancement vs. soft tissue kernels, pitch, field of view (FOV), pixel size, and focal spot size, among others.

Various other metrics may be used to assess image quality. For many applications, a valuable quality metric correlates well with the subjective perception of quality by a human observer. One type of metrics, referred to as full-reference metrics, include comparing a first version of an image including distortions with a second, reference version of the image not including distortions. These metrics may include, for example, SNR, various structural similarity (SSIM) metrics, NPS, e.g., an intensity of noise as a function of spatial frequency (noise texture); visual information fidelity (VIFp), feature similarity index measurement (FSIM), spectral residual-based similarity (SR-SIM), gradient magnitude similarity deviation (GMSD), visual saliency-induced index (VSI), and deep image structure and texture similarity (DISTS), among others.

If no reference versions of the image are available, various non-reference metrics and/or models may be used to estimate a quality of an image based on expected image statistics. Examples of non-reference models include a natural image quality evaluator (NIQE) model, which is based on measuring deviations from statistical regularities in natural images; a perception-based image quality evaluator (PIQE) model, which is based on mean subtraction contrast normalization; and a Blind/Referenceless Image Spatial Quality Evaluator (BRISQUE) model, which is a scene statistic model based on locally normalized luminance coefficients of an image.

Objective image quality assessment methods may be based on various computed low-level metrics used to determine image quality, including SNR, CNR, SSIM, uniformity metrics, and others. SNR and peak SNR (PSNR) are based on capturing signal visibility drawn in noise on a noise map, where a higher (P)SNR is an indicator of higher image quality. SSIM is computed by comparing an image to a denoised (reference) image. Alternatively, a histogram flatness measure (HFM) and/or a histogram spread (HS) capture variations in contrast in an image, where a low HFM/HS value indicates low contrast in the image, and a high HFM/HS value indicates high contrast in the image.

To generate high quality CT images, various parameters of a CT system may be initially set at the beginning of a scan, where settings of the various parameters are established by a scan protocol of the scan. The established parameter settings may be established in the scan protocol by one or more human experts, based on experience, research, and/or predictive models (statistical models, AI/ML models, etc.), or the established parameter settings may be generated by the predictive models without the involvement of human experts. The predictive models may be trained using training data including a plurality of acquisition parameter settings and high quality CT images generated using the acquisition parameter settings as ground truth data. However, as collecting the training data may be cumbersome and time consuming, available training data may be insufficient.

As a result, the parameter settings established in the scan protocol may not generate images of high quality, and the parameters may be adjusted by an operator of the CT system, before or during the scan. For example, a scanned subject may be larger than a typical scanned subject, and the operator may adjust one or more parameters based on the larger size of the scanned subject. If an amount of noise observed in a reconstructed image is higher than desired, a radiation dosage applied by the CT system may be increased. If a spatial resolution of the image is lower than desired, a size of a focal spot of the CT system may be decreased, and/or an FOV of the CT system may be decreased, or a different parameter may be adjusted. Thus, generating high quality CT images may depend on first assessing the quality of a reconstructed image, and based on the assessment, setting specific parameters of the CT system to achieve a desired quality.

The predictive models used by the one or more human experts to generate recommended parameter settings for scan protocols and/or recommendations for adjusting the parameter settings of the scan protocols may be periodically adjusted and/or retrained, which may result in improved recommended settings that result in images of increased quality. However, generating image quality data suitable for retraining the models may rely on efficiently collecting image quality assessment feedback from a plurality of operators of the CT system. For the image quality data to be suitable, a desired specificity of the feedback may be high. For example, an image quality assessment for an ROI may be different from an image quality assessment of a slice of the image including the ROI, which may be different from an image quality assessment of the entire image. Additionally or alternatively, an image quality of a first ROI of an image may be different from an image quality of a second ROI of the image.

To efficiently collect data with this degree of specificity, the inventors herein have developed an assessment tool for automatically evaluating image quality of medical images, and collecting operator feedback with respect to the image quality via a proposed image quality graphical user interface (GUI). The tool assesses noise, texture, contrast, resolution, and other characteristics of slices of an image, anatomical regions of the image, and/or ROIs of the image based on various low-level metrics, and calculates an overall image quality score of the image by aggregating the low-level metrics. Specifically, the score may be estimated by a machine learning (ML) model based on the low-level metrics. The user can provide feedback with respect to the image quality score, which may be used to increase an accuracy and/or performance of the ML model. In this way, feedback may be efficiently collected and used to refine the ML model, leading to more accurate scan protocols, higher quality CT images, and better patient outcomes.

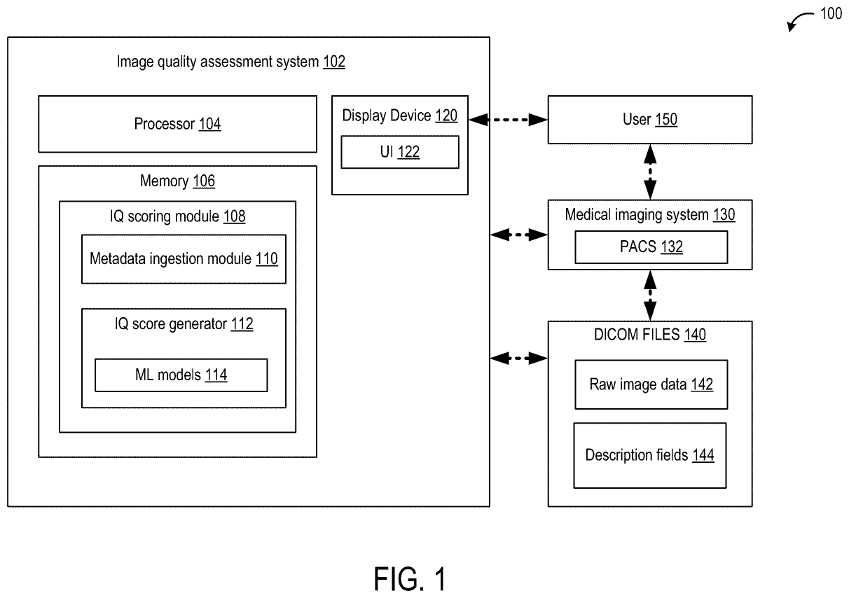

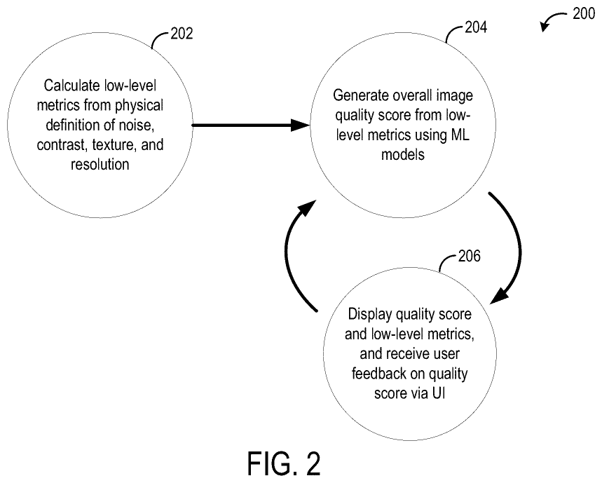

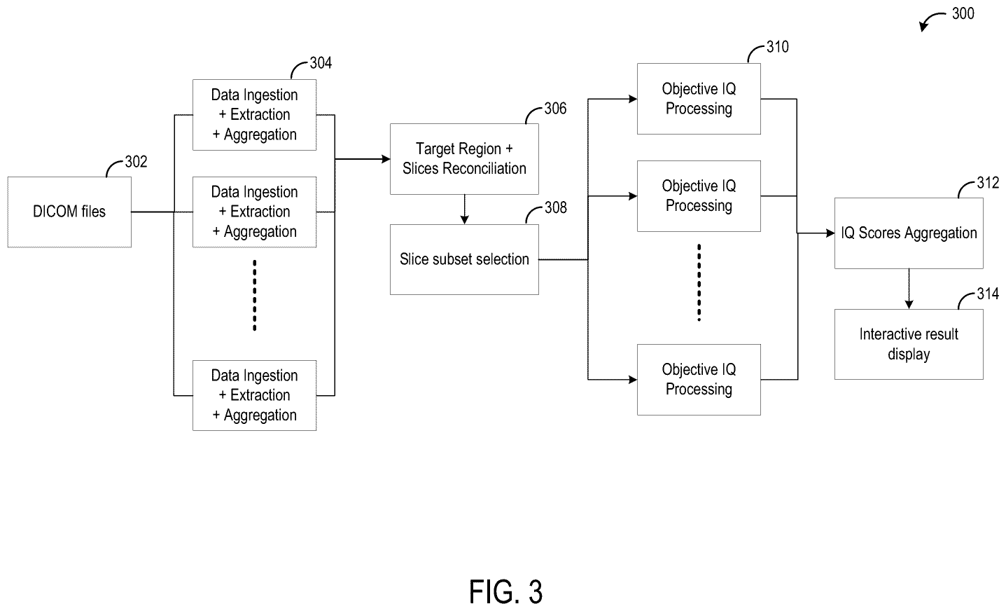

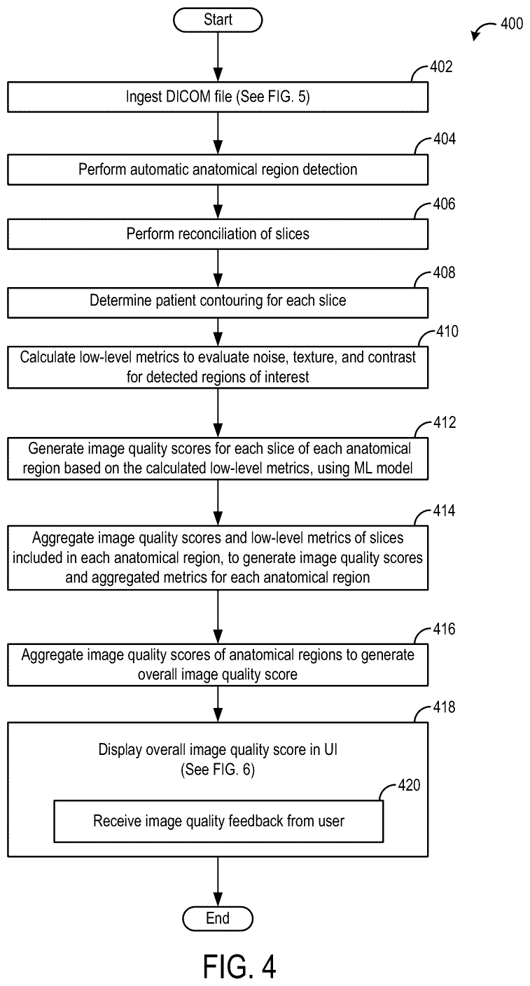

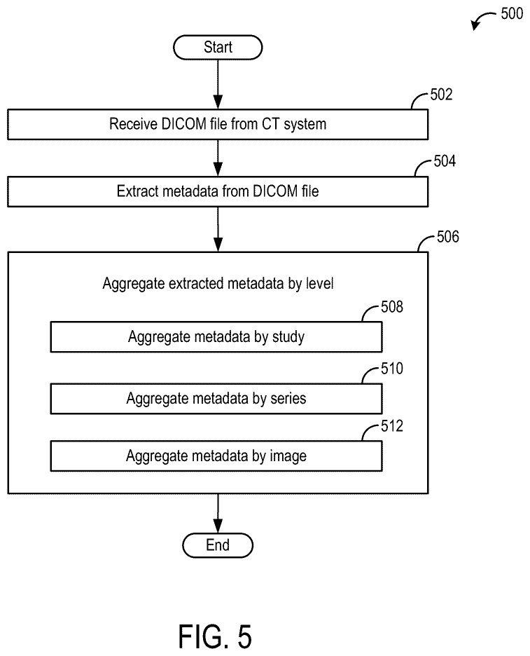

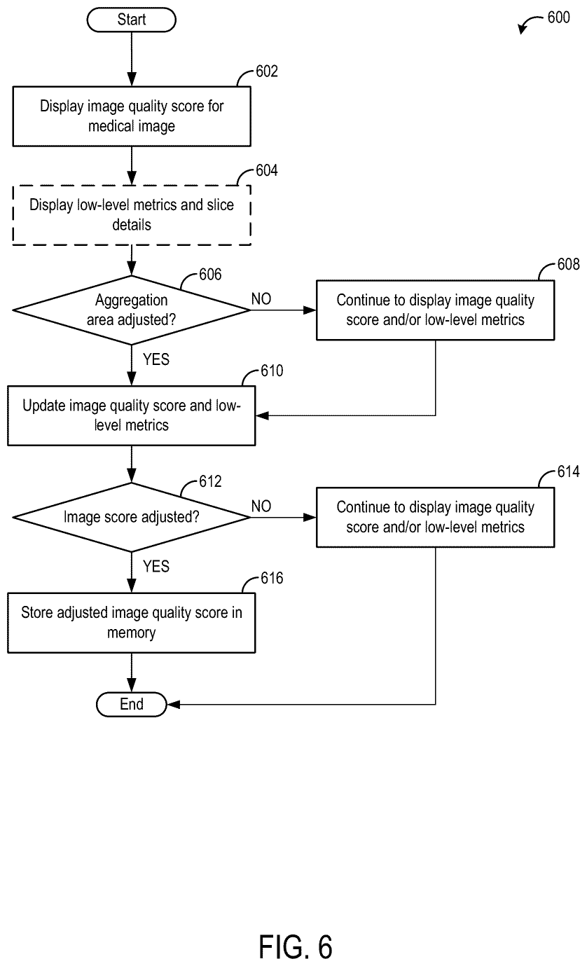

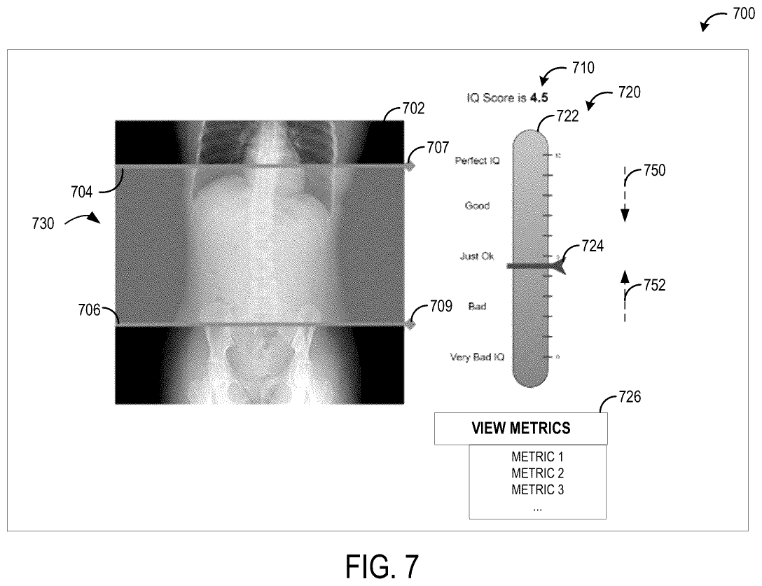

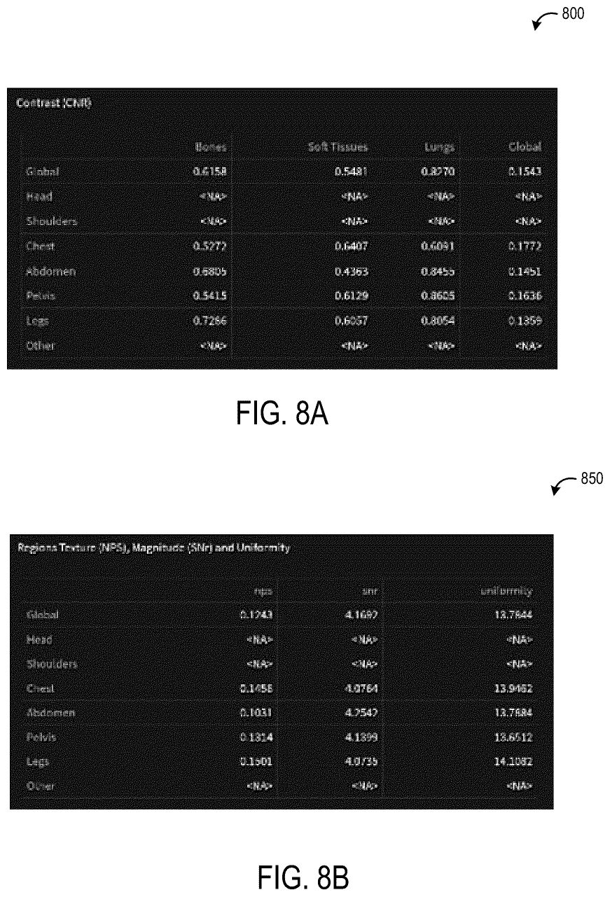

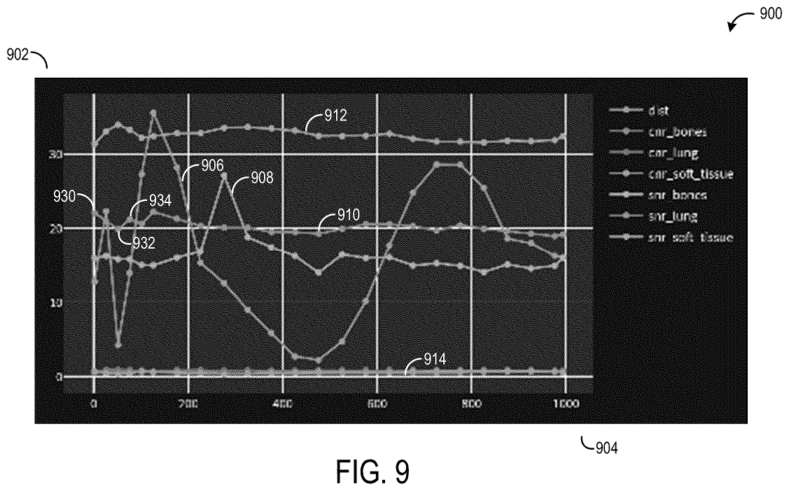

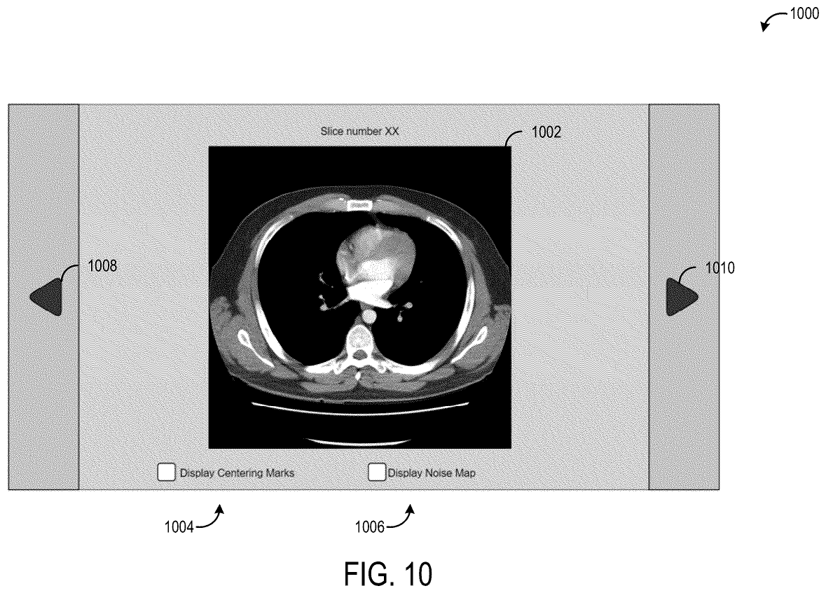

FIG. 1 shows an image quality assessment system, including an image quality scoring module that may be used to automatically score a quality of an image reconstructed from projection data acquired using a medical imaging system, in accordance with a strategy indicated in FIG. 2. The strategy may be implemented by following the general procedure shown in FIG. 3. The general procedure may be carried out by following one or more steps of a method shown in FIG. 4, which may include processing a set of DICOM files of a medical imaging exam, as described in relation to the method shown in FIG. 5, and receiving feedback from a user of the image quality assessment system, as described in relation to the method shown in FIG. 6. An image quality score resulting from following the method may be displayed on a display device in an image quality GUI of the image quality assessment system shown in FIG. 7. The user may additionally view selectable, abbreviated representations of low-level metrics used to generate the image quality score in one or more display panels of the image quality GUI, as shown in FIGS. 8A and 8B, and/or a graph showing selectable, summarized low-level metric data, as shown in FIG. 9. An individual 2-D slice image may be displayed, as shown in FIG. 10, for example, when the user selects a point on the graph of FIG. 9.

Referring to FIG. 1, a medical system 100 is shown comprising an image quality assessment system 102, a medical imaging system 130, and a user 150, where user 150 may be a user of image quality assessment system 102, medical imaging system 130, or both image quality assessment system 102 and medical imaging system 130. In some embodiments, image quality assessment system 102 may be included in medical imaging system 130. For example, image quality assessment system 102 may be included in an image processing unit of medical imaging system 130, where the image processing unit is configured to reconstruct images of a subject using an iterative or analytic image reconstruction method, such as filtered back projection (FBP), advanced statistical iterative reconstruction (ASIR), conjugate gradient (CG), maximum likelihood expectation maximization (MLEM), model-based iterative reconstruction (MBIR), or a different reconstruction method. In other embodiments, image quality assessment system 102 may not be included in medical imaging system 130, and image quality assessment system 102 may be coupled to medical imaging system 130 via a wired or wireless connection, such that data may be transmitted between medical imaging system 130 and image quality assessment system 102.

In some embodiments, medical imaging system 130 either includes, or is coupled to, a picture archiving and communications system (PACS) 132. In an exemplary implementation, the PACS 132 is further coupled to a remote system such as a radiology department information system, hospital information system, and/or to an internal or external network (not shown) to allow operators at different locations to supply commands and parameters and/or gain access to the image data.

Image quality assessment system 102 may be a computing device, such as a desktop computer (e.g., a PC or a workstation), a laptop, a tablet, or different kind of computing device. Image quality assessment system 102 may be an image processing device dedicated to reviewing images from medical exams, such as an image review server, an image post-processing system, a PACS system (e.g., PACS 132), an acquisition console on an acquisition machine, or a cloud multi-tenant image review service.

Medical images acquired via medical imaging system 130 during a medical imaging exam may be stored in accordance with a Digital Imaging and Communications in Medicine (DICOM) standard, and image quality assessment system 102 may receive images from one or more DICOM files 140. The DICOM standard defines a file format that includes various fields for information that an image processing application may use to display and/or preprocess imaging data included in a DICOM file. DICOM files 140 may include raw image data 142 acquired during the medical imaging exam. DICOM files 140 may also include a plurality of description fields 144, where each description field 144 may include one or more expressions relevant to the medical imaging exam, an acquisition protocol of the exam, a series of the exam and/or images of the exam. The expressions may be single words, or multi-word expressions. For example, the expressions may include an anatomy of a patient; contrast information, including a contrast phase and a contrast agent; an acquisition gating; a laterality of raw image data 142; a pathology detected in raw image data 142; reconstruction filters used; a multi-energy indication; a weighting and/or pulse sequence, in the case of magnetic resonance (MR) images; and/or other options selected during the medical exam.

Image quality assessment system 102 may include a processor 104, a memory 106, and a display device 120. Processor 104 may control an operation of image quality assessment system 102, and may receive control signals from user 150 via display device 120. Display device 120 may include a display (e.g., screen or monitor) and/or other subsystems. In some embodiments, display device 120 may be integrated into image quality assessment system 102, where a user may interact with, adjust, or select control elements in the display device 120 (e.g., buttons, knobs, touchscreen elements, etc.) to send one or more control signals to the processor 104 from display device 120. In other embodiments, display device 120 is not integrated into the image quality assessment system 102, and the user may interact with, adjust, or select control elements in display device 120 via a user input device, such as a mouse, track ball, touchpad, etc., or the operator may interact with display device 120 via a separate touchscreen, where the operator touches a display screen of display device 120 to interact with image quality assessment system 102, or via another type of input device.

The operator may interact with image quality assessment system 102 via an image quality graphical user interface (GUI) 122. In some embodiments, image quality GUI 122 may be integrated into a GUI of a different system, such as medical imaging system 130. For example, various medical exam reviewing applications may be installed on medical imaging system 130 for viewing, reviewing, navigating through, and/or analyzing images of a medical exam. Each medical exam reviewing application may be suitable and/or preferred for a different type of medical exam. Each medical exam reviewing application may include a corresponding GUI for reviewing a medical exam, where image quality GUI 122 may be integrated into the corresponding GUI.

Processor 104 may execute instructions stored on the memory 106 to control image quality assessment system 102. Processor 104 may be single core or multi-core, and the programs executed thereon may be configured for parallel or distributed processing. In some embodiments, the processor 104 may optionally include individual components that are distributed throughout two or more devices, which may be remotely located and/or configured for coordinated processing. In some embodiments, one or more aspects of the processor 104 may be virtualized and executed by remotely-accessible networked computing devices configured in a cloud computing configuration.

As discussed herein, memory 106 may include any non-transitory computer readable medium in which programming instructions are stored. For the purposes of this disclosure, the term “tangible computer readable medium” is expressly defined to include any type of computer readable storage. The example methods and systems may be implemented using coded instruction (e.g., computer readable instructions) stored on a non-transitory computer readable medium such as a flash memory, a read-only memory (ROM), a random-access memory (RAM), a cache, or any other storage media in which information is stored for any duration (e.g. for extended period time periods, permanently, brief instances, for temporarily buffering, and/or for caching of the information). In some embodiments, the non-transitory computer readable medium may be distributed across various computers and/or servers (e.g., provided via web services). Computer memory of computer readable storage mediums as referenced herein may include volatile and non-volatile or removable and non-removable media for a storage of electronic-formatted information such as computer readable program instructions or modules of computer readable program instructions, data, etc. that may be stand-alone or as part of a computing device. Examples of computer memory may include any other medium which can be used to store the desired electronic format of information and which can be accessed by the processor or processors or at least a portion of a computing device. In various embodiments, memory 106 may include an SD memory card, an internal and/or external hard disk, USB memory device, or similar modular memory.

Program code embodied on a computer readable medium may be transmitted using any appropriate medium, including but not limited to wireless, wireline, optical fiber cable, RF, etc., or any suitable combination of the foregoing. Computer program code for carrying out operations for aspects of the present disclosure may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Memory 106 may store an image quality scoring module 108 that comprises instructions for generating an image quality score of a medical image, as disclosed herein. An overall procedure for generating the image quality score is shown in FIG. 3. Specifically, image quality scoring module 108 may include instructions that, when executed by processor 104, cause the image quality assessment system 102 to conduct one or more of the steps of method 400, as described in further detail below.

Image quality scoring module 108 may include various modules including instructions for performing various tasks involved in generating a quality score for the medical image. Image quality scoring module 108 may include a metadata ingestion module 110 that comprises instructions for ingesting image data from one or more DICOM files 140. The image data may include metadata stored in one or more of the description fields 144 of the one or more DICOM files 140. The ingestion of the image data performed with the metadata ingestion module 110 may include extracting and aggregating the metadata, as described in greater detail in reference to FIG. 5. In particular, metadata ingestion module 110 may include instructions that, when executed by processor 104, cause the image quality assessment system 102 to conduct one or more of the steps of method 500 of FIG. 5, as described in further detail below.

Image quality scoring module 108 may include an image quality score generator 112. In various embodiments, image quality score generator 112 may include one or more ML models 114, which may be trained to generate (e.g., estimate) an image quality score for an image received from DICOM files 140. One advantage of using image data included in DICOM files for training the one or more ML models 114 is that the image data is independent of an acquisition system's vendor and/or model, and may be generated from various proprietary systems. As a result, it is possible to use image data from multiple systems and/or sites to train an ML model, whereby an increased amount of data may be collected in a shortened time frame. Generation of the image quality score is described in greater detail below in reference to FIG. 4. An example of a GUI including the image quality score is shown in FIG. 7.

FIG. 2 shows a general strategy for generating an image quality score for a medical image, such as a CT image reconstructed from projection data acquired via a CT system, using an image quality assessment system such as medical imaging processing system 101 of FIG. 1. The general strategy depicted in FIG. 2 includes 3 stages. In a first stage 202, low-level metrics are calculated for each slice of the medical image, from a physical definition of noise, contrast, and texture. At a second stage 204, an image quality score for the medical image is generated from the low-level metrics by an image quality score generator (e.g., image quality score generator 112), using an ML model (e.g., an ML model 114). For example, the low-level metrics may be calculated for each slice of the image, and the low-level metrics may be inputs into the ML model, where the image quality score is an output of the ML model. The outputs of the ML model for each slice may be aggregated across the slices (and/or anatomical regions and/or ROIs associated with the slices) of the medical image, to generate the image quality score. At a third stage 206, the image quality score and the low-level metrics used to generate the image quality score are displayed to a user of the image quality assessment system. The user may provide feedback regarding the image quality score, which may be collected, stored, and used to refine or retrain the one or more ML models to increase an accuracy of the image quality score.

An additional advantage of the methods and systems described herein is that the user can select a portion of the medical image (for example, a portion including a desired anatomical region or ROI), and view an image quality score for the selected portion, which may be different from the image quality score for the entire image and/or image quality scores of other portions of the medical image. For example, the portion may be selected by adjusting one or more repositionable boundaries defining an area of the medical image over which the low-level metrics are aggregated to generate a corresponding image quality score. In other words, a first selected aggregation area may result in a first image quality score based on low-level metrics calculated from slices included in the first selected aggregation area, where the first image quality score is not based on low-level metrics calculated from slices outside the first selected aggregation area. The user may adjust the repositionable boundaries of the first selected aggregation area to create a second selected aggregation area. The second selected aggregation area may result in a second image quality score based on low-level metrics from slices included in the second selected aggregation area, where the second image quality score is not based on low-level metrics calculated on slices outside the second selected aggregation area.

When the user adjusts the repositionable boundaries of the first aggregation area to create the second aggregation area, the image quality score generator may not be relaunched to recalculate the image quality score for the second aggregation area. Rather, slices included within the repositionable boundaries of the second aggregation area are selected, and the image quality score for the second aggregation area may be calculated by aggregating the image quality scores generated for each individual slice of the selected slices. In this way, image quality scores for different selected aggregation areas may be displayed in the GUI more rapidly than if the image quality score generator were relaunched each time an aggregation arca is changed by the user. As a result, in an amount of time available to the user to view the medical image, the user may provide feedback with respect to the image quality score displayed for the medical image, and additionally specify one or more portions (e.g., anatomical regions or ROIs) of the medical image, view image quality scores corresponding specifically to the portions, and provide feedback on the image quality scores of the portions. By reducing an amount of time taken by the user to provide feedback on the medical image and/or the portions of the medical image, an amount of feedback data collected by the image quality assessment system may be increased. By increasing the amount of feedback data collected by the image quality assessment system, an amount of training data used to retrain the ML model may be increased, thereby increasing the accuracy and/or performance of the ML model. Thus, a virtuous cycle may be created, leading to increasingly accurate image quality scores and as a result, higher quality images.

FIG. 3 shows an overview of stages of a high-level procedure for generating the medical image quality score within the image quality assessment system. The stages illustrated in FIG. 3 are described in greater detail below in reference to the methods of FIGS. 4, 5, and 6.

In a first stage, image data from a DICOM file 302 is ingested into the image quality assessment system during a data ingestion process 304. During ingestion, metadata included in DICOM file 302 may be extracted and aggregated, as described below in reference to FIG. 5.

In a second stage, a slice reconciliation process 306 may be performed, where one or more anatomical regions of the image may be identified, and each slice of the image may be associated with an anatomical region of the one or more anatomical regions. Identification of the one or more anatomical regions may be performed using a localizer image, such as an anterior-posterior (AP) localizer image or a lateral (LAT) localizer image where each slice is related to a position on the localizer image to determine which anatomical region the slice belongs to. Additionally, during slice reconciliation process 306, slices of the image may be associated with an ROI within an anatomical region of the image, where the ROI may be specified by the user via a GUI of the image quality assessment system.

In a third stage, a subset of the slices may be selected during a slice subset selection process 308, and the subset of slices may be processed to determine a set of image quality scores during an image quality score generation process 310, as described in detail below in reference to FIG. 4. In a fourth stage, during an image quality score aggregation process 312, the set of image quality scores may be aggregated, and the aggregated results may be displayed to a user of the image quality assessment system at an interactive result display step 314, as described in detail below in reference to FIG. 6.

Referring now to FIG. 4, an exemplary method 400 is shown for generating and displaying a quality score for an image, such as a CT image, reconstructed from a medical imaging exam. In various embodiments, the image may be stored in a DICOM file (e.g., the DICOM files 302 of FIG. 3). Method 400 and other methods described herein are described with reference to an image quality assessment system, such as image quality assessment system 102 of FIG. 1, and in particular, an image quality scoring module of the image quality assessment system. Method 400 and the other methods described herein may be implemented via computer-readable instructions stored in a memory of an image quality assessment system, and executed by a processor of the image quality assessment system, such as memory 106 and processor 104 of image quality assessment system 102 of FIG. 1.

Method 400 begins at 402, where method 400 includes ingesting a DICOM file corresponding to the image. Ingestion of the DICOM file is described in greater detail in FIG. 5.

Referring to FIG. 5, an exemplary method 500 is shown for ingesting and processing a DICOM file, such as a DICOM file 140 of FIG. 1, via an image quality assessment system (e.g., image quality assessment system 102). Method 500 begins at 502, where method 500 includes receiving a DICOM file. In some embodiments, the DICOM file may be received from a CT system (e.g., medical imaging system 130) electronically coupled to the image quality assessment system, where the DICOM file corresponds to an image acquired by the CT system. The DICOM file may be received at a time of a scan performed by an operator of the CT system, or the DICOM file may be received after a scan is performed.

At 504, method 500 includes extracting metadata from the DICOM files. In various embodiments, the metadata may be extracted and aggregated by level (e.g., study, series, image, etc.) For example, in a first step, metadata pertaining to a study of the DICOM file may be extracted from one or more description fields (e.g., description fields 144 of FIG. 1) of the DICOM file. In a second step, metadata pertaining to a series of the DICOM file may be extracted from the one or more description fields. In a third step, metadata pertaining to an image of the DICOM file may be extracted from the one or more description fields, and so on.

At 506, method 500 includes aggregating the extracted metadata by level. At 508, aggregating the extracted metadata by level includes aggregating the metadata collected with respect to a study of the DICOM file. At 510, aggregating the extracted metadata by level includes aggregating the metadata collected with respect to a series of the DICOM file. At 512, aggregating the extracted metadata by level includes aggregating the metadata collected with respect to an image included in the DICOM file. Method 500 ends.

Returning to FIG. 4, at 404, method 400 includes performing automatic detection of one or more anatomical regions of the image. In various embodiments, the automatic anatomical region detection is performed using one or more localizer images, such as an AP localizer image and/or a LAT localizer image. The localizer image may be generated before or at a time of acquisition via a CT system, and may be included in one or more DICOM files associated with the image. In one embodiment, a location of a slice of the image may be determined from DICOM metadata. For example, the location may be referenced by a Slice Location DICOM tag.

At 406, method 400 includes performing reconciliation of slices. During the reconciliation of slices, each slice of a given series may be reconciled to a position on the localizer image. The position of the slice may be projected on a Z-axis of the localizer image. When the position of the slice is matched to the localizer image, the anatomical region the slice belongs to can be determined. Thus, a result of the slice reconciliation process is an association of various sets of slices with corresponding detected anatomical regions.

In some embodiments, slice reconciliation may be performed on a specific ROI within a detected anatomical region. For example, a user may specify an ROI via a GUI of the image quality assessment system (e.g., image quality GUI 122), and perform the slice reconciliation on the specified ROI. FIG. 7 shows an exemplary graphical element that may allow the user to select an ROI in an image.

At 408, method 400 includes automatically determining patient contouring for each slice. The patient contouring includes differentiating between pixels of the image that belong to the scanned subject, and pixels of the image outside the scanned subject. During the patient contouring, the pixels belonging to the scanned subject are selected, and the pixels not belonging to the scanned subject are not selected. In this way, we exclude pixels that represent air around the scanned subject, pixels representing the table, and/or other artefacts that might appear in the image. An image quality assessment is subsequently performed on the selected pixels, and not performed on pixels that are not selected.

At 410, method 400 includes calculating low-level metrics to evaluate noise, texture, and contrast in each slice of each detected anatomical region (and/or specified ROI). The low-level metrics may include, for example, an overall noise level and/or SNR, which may be further broken down into one or more noise levels and/or SNRs of soft tissues, bones, and/or an ROI; an overall CNR, which may be further broken down into one or more CNRs of soft tissues, bones, and/or an ROI; a uniformity metric; a maximum, minimum, and/or mean noise power spectrum (NPS) value; a noise level; and so on. It should be appreciated that the examples provided herein are for illustrative purposes, and other low-level metrics may be included without departing from the scope of this disclosure.

At 412, method 400 includes generating image quality scores for each slice of each detected anatomical region based on the calculated low-level metrics, using an ML model (e.g., an ML model 114). For example, a plurality of numerical values obtained from the calculated low-level metrics may be inputs into the ML model, and the ML model may be trained to predict an image quality score based on the plurality of numerical values. In various embodiments, the ML model is trained using training data including low-level metrics of training images, and ground truth image quality scores of the training images determined by human experts. In other embodiments, different training data may be used.

At 414, method 400 includes aggregating the image quality scores and low-level metrics of the slices included in each detected anatomical region, to generate image quality scores and aggregated metrics for the detected anatomical regions. For example, aggregating the image quality scores of the slices included in each detected anatomical region may include calculating an average of the image quality scores of the slices, or a different function may be used to aggregate the image quality scores. Similarly, the low-level metrics used to generate the image quality scores for the slices of a detected anatomical region may be aggregated, such that a single numerical value corresponding to a low-level metric may be obtained for the detected anatomical region. The single numerical value corresponding to the low-level metric may be displayed in the GUI, so that a user can see how an image quality score was generated.

At 416, method 400 includes aggregating the image quality scores of the detected anatomical regions, to generate an overall image quality score for the image. In various embodiments, aggregating the image quality scores of the detected anatomical regions may include calculating an average of the image quality scores of the detected anatomical regions, or a different function may be used to aggregate the image quality scores.

At 418, method 400 includes displaying the overall image quality score in a GUI of the image quality assessment system (e.g., image quality GUI 122). Displaying the image quality score is described in greater detail below in reference to FIG. 6. At 420, method 400 includes receiving image quality feedback from a user of the image quality assessment system, and method 400 ends.

As an example of how method 400 might be applied, an operator of a CT system may perform a CT scan on a patient. A 3-D CT image acquired by the CT system may be stored in a DICOM file in a memory of the CT system, or in a different system coupled to the CT system. A radiologist may wish to view the 3-D CT image, for example, to perform a diagnosis of the patient. The radiologist may load the DICOM file into the image quality assessment system to view the 3-D CT image. The image quality assessment system may ingest the DICOM file, and extract metadata associated with the 3-D CT image. The image quality assessment system may use the extracted metadata to detect various anatomical regions of the CT image, using a localizer image, and reconcile 2-D slices of the CT image with the anatomical regions, as described above. As a result of the slice reconciliation process, various sets of slices may be associated with each detected anatomical region. The image quality assessment system may perform a patient contouring process to eliminate image data not corresponding to the patient (e.g., outside the body of the patient) from each slice.

For each 2-D slice of the 3-D CT image, low-level image quality metrics may be calculated that may be indicative of a quality of the 2-D slice. Additionally, low-level image quality metrics may be calculated for one or more detected anatomical regions within the 2-D slice, which may be indicative of a quality of portions of the 2-D slice corresponding to the detected anatomical regions. Results of the low-level image quality metrics for the 2-D slice may be inputted into an ML model of an image quality score generator, which may generate an image quality score of the 2-D slice. Additionally, results of the low-level image quality metrics for the one or more detected anatomical regions may be inputted into the ML model to generate image quality scores for the portions of the 2-D slice corresponding to the detected anatomical regions.

Image quality scores for the detected anatomical regions may be calculated, where the image quality score of a detected anatomical region may be a function of (e.g., an average of) the image quality scores of each of the 2-D slices associated with the detected anatomical region during the slice reconciliation. Additionally, the results of the low-level image quality metrics used to generate the image quality scores of each of the 2-D slices associated with the detected anatomical region may be aggregated across the 2-D slices to generate aggregate (e.g. average) low-level metric data for the detected anatomical regions.

The image quality assessment system may then generate an overall image quality score for the 3-D CT image, as a function of the image quality scores of the 2-D slices included in the 3-D CT image. Additionally or alternatively, the overall image quality score may be generated as a function of the image quality scores of the detected anatomical regions.

The image quality assessment system may display the 3-D CT image and/or the 2-D slices in a GUI of the image quality assessment system, to be viewed by the radiologist. For each image the radiologist views, the image quality assessment system may display the overall image score associated with the image. In various embodiments, the overall image score may be displayed in an interactive graphical element, as described below in reference to FIG. 7. If the radiologist disagrees with an image quality score assigned to an image, the radiologist may adjust the image quality score to a more suitable image quality score, using the interactive graphical element. For example, if the radiologist feels that the image quality score is too high, the radiologist may reduce the image quality score, and if the radiologist feels that the image quality scores too low, the radiologist may increase the image quality score. When the image quality score is adjusted, the adjusted image quality score may be saved by the image quality assessment system. The saved, adjusted image quality score may be included in training data of the ML model, where the ML model may be retrained or refined in a subsequent training stage using the training data. In this way, an accurate set of ground truth training data based on human expert opinions may be obtained in an efficient manner, for example, from a plurality of radiologists, thereby increasing in accuracy and/or a performance of the ML model.

Referring now to FIG. 6, an exemplary method 600 is shown for displaying image quality scores associated with a medical image to a user of an image quality assessment system, such as image quality assessment system 102 of FIG. 1, in a GUI of the image quality assessment system, and receiving feedback from the user regarding an accuracy of the image quality score. The image quality scores may be generated via a method such as method 400 described above in reference to FIG. 4.

Method 600 begins at 602, where method 600 includes displaying an image quality score generated for the medical image. As described above, the image quality score may be an aggregate score that aggregates image quality scores generated from slices of the medical image. Additionally, image quality scores and low-level image quality metric data associated with one or more anatomical regions (head, shoulders, pelvis, abdomen, etc.) of the medical image, and/or one or more ROIs (e.g., tissues, bones, organs, etc.) of the image may be advantageously displayed, without regenerating the image quality scores of the slices, by selecting control elements in an image quality GUI, such as the an image quality GUI shown in FIG. 7.

Turning briefly to FIG. 7, an image quality GUI 700 of an image quality assessment system is shown, such as image quality GUI 122 of image quality assessment system 102. Image quality GUI 700 may be displayed on a display device of an image quality assessment system, such as display device 120 of image quality assessment system 102. Image quality GUI 700 includes a reference image 702, where reference image 702 is a 2-D image of a scanned subject showing a series scanned area of a 3-D medical image (also referred to herein as the medical image) acquired via a medical imaging system (e.g., medical imaging system 130 of FIG. 1). Image quality GUI 700 includes an image quality score 710 indicating a quality of the medical image, specifically, to an overall quality of slices of the medical image included in the series scanned area. In various embodiments, image quality score 710 is a numeric value calculated as described above in reference to FIG. 4. Image quality score 710 may be displayed within a textual context, as shown in image quality GUI 700, or image quality score 710 may be displayed in a different manner, such as within a graphical element of image quality GUI 700.

Image quality GUI 700 includes an image quality score indicator 720, on which image quality score 710 is indicated. In the embodiment depicted in FIG. 7, image quality score indicator 720 is a slider 722 showing a range of image quality score descriptors (e.g., perfect, good, ok, bad, etc.), where the image quality score is indicated by a needle 724 of slider 722. In other embodiments, a different graphical element may be used to indicate the image quality score 710, such as, for example, a dial.

Image quality score indicator 720 may be an interactive graphical element, where a user of the GUI may adjust a relative position of needle 724 within slider 722 to change image quality score 710. For example, the user may not agree with the image quality score, and the user may wish to adjust the image quality score to a more accurate image quality score. For example, the user may feel that the image quality is higher than indicated by the image quality score, and the user may adjust needle 724 upward in a direction 752 to increase the image quality score, or the user may feel that the image quality is lower than indicated by the image quality score, and the user may adjust needle 724 downward in a direction 750 to decrease the image quality score. In various embodiments, when the user adjusts the image quality score in image quality score indicator 720, the adjusted image quality score is stored in a memory of the image quality assessment system (e.g., memory 106). The adjusted image quality score may be included in training data used to retrain an ML model used to generate the image quality score (e.g., at a later stage), to increase an accuracy of the image quality score.

Image quality GUI 700 may include a menu 726, which may include, as menu items, a limited list of low-level metrics used to generate image quality score 710. For example, the limited list of low-level metrics may include an SNR, a CNR, an NPS, etc. In response to the user selecting a menu item corresponding to a low-level metric of the limited list of low-level metrics, a display panel (e.g., a pop-up) of the GUI may be displayed, showing one or more summaries of results of the low-level image quality metric. The one or more summaries may include tables of low-level image quality metric results, graphs of low-level image quality metric results, and/or images of slices of the medical image. Examples of the one or more summaries are shown below in FIGS. 8A, 8B, 9, and 10.

In some examples, menu 726 may not be a menu element, and menu 726 may include one or more checkboxes of items, such that a plurality of low-level metrics may be selected, or a different type of selection element.

In some embodiments, a display panel may be displayed that can be reached directly from the menu that shows a preview of data included in a summary of the one or more summaries. For example, when the user selects menu 726, a first display panel may be displayed showing a limited list of low-level image quality metric results (e.g., such as FIG. 8A or 8B), where the results are individually or collectively selectable. The user may view the limited list to determine a specific low-level image quality metric to view. The user may then select an item of the limited list corresponding to the specific low-level image quality metric. When the menu item is selected, a second display panel may be displayed showing a graph of the specific low-level image quality metric (e.g., such as FIG. 9). Alternatively, when the user selects menu 726, a first display panel may be displayed showing a graph of a plurality of low-level image quality metric results (e.g., FIG. 9). The user may view the graph to determine a specific low-level image quality metric to view. The user may then select a menu item of menu 726 corresponding to the specific low-level image quality metric. When the menu item is selected, a second display panel may be displayed showing a list of results of the specific low-level image quality metric (e.g., such as FIG. 8A or 8B).

In this way, the user may view an abbreviated representation of data to determine which specific data to view in a subsequent summary display. By previewing the abbreviated representation in the first display panel, an amount of time spent by the user determining which data to view may be reduced, making image quality GUI 700 more efficient. Further, computational and memory resources of the image quality assessment system may be used in a more efficient manner, improving the functioning of the image quality assessment system and increasing capabilities of the image quality assessment system.

Additionally, an aggregation area 730 may be displayed in a superimposed fashion on reference image 702, where aggregation area 730 may initially be the series scanned area (e.g., including all the slices of the medical image). In the embodiment depicted in FIG. 7, aggregation area 730 is defined by a position of an upper horizontal boundary 704 and a lower horizontal boundary 706. In various embodiments, upper horizontal boundary 704 and lower horizontal boundary 706 are interactive graphical elements that may be repositioned by the user. For example, the user may reposition upper horizontal boundary 704 by selecting an upper handle 707, and sliding upper handle 707 vertically in direction 750 or direction 752. Similarly, the user may reposition lower horizontal boundary 706 by selecting a lower handle 709, and sliding lower handle 709 vertically in direction 750 or direction 752. As upper horizontal boundary 704 and lower horizontal boundary 706 are repositioned (e.g., using handles 707 and/or 709), a size of aggregation area 730 may increase or decrease.

Returning to FIG. 6, at 604, method 600 may include displaying low-level metrics and slice details. The low-level metrics and slice details may be displayed via display panels, such as the exemplary display panels shown in FIGS. 8A, 8B, 9, and 10.

FIG. 8A shows an exemplary display panel 800 indicating CNRs calculated for the medical image and various anatomical regions of the medical image. For each region, a first CNR is displayed for portions of bone within the region; a second CNR is displayed for portions of soft tissues within the region; a third CNR is displayed for portions of a selected ROI of the medical image; and a fourth, global CNR is displayed for each anatomical region.

FIG. 8B shows an exemplary display panel 850 indicating an NPS (e.g., a texture), an SNR, and a uniformity calculated for the medical image and various anatomical regions of the medical image.

FIG. 9 shows a graph 900, indicating an exemplary overview of low-level metrics across a plurality of series of the medical image. Graph 900 includes a vertical axis 902, indicating a range of SNR values, and a horizontal axis 904, indicating a progression of slides of the medical image. Thus, lines on graph 900 may show various low-level metrics for each slide of the progression of slides, and how the various low-level metrics change over the progression of slides. Graph 900 includes a first line 906, indicating results of a first low-level metric; a second line 908, indicating results of a second low-level metric, a third line 910, indicating results of a third low-level metric; and a fourth line 912, indicating results of a fourth low-level metric. The results may correspond to one or more ROIs. Each point indicated on lines 906, 908, 910, and 912 represents a slice of the progression of slices. For example, a point 930 indicates a first slice of line 910; a point 932 indicates a second slice of line 910; a point 934 indicates a third slice of line 910; and so on. A fifth line 914 shows a distribution of the slices.

In various embodiments, graph 900 may be displayed as a result of the user selecting elements of data displayed on the display panels 800 and/or 850. For example, the user may select the ROI “lungs” of display panel 800, and in response, the image quality assessment system may generate graph 900 displaying the low-level image quality metric CNR for slides associated with lungs of a patient of the image. The user may select a plurality of ROIs, and/or a plurality of low-level metrics, and the image quality assessment system may generate a corresponding plurality of lines on graph 900.

Graph 900 may be an interactive graph, and points of the graph including points 930, 932, 934 may be selectable by a user. When the user selects a point on the graph, the corresponding slice may be displayed in the GUI. An example of a slice corresponding to a point is shown in FIG. 10.

FIG. 10 shows an exemplary display panel 1000 showing a 2-D slice 1002 of a medical image. In various embodiments, 2-D slice 1002 may be displayed as a result of a user of an image quality assessment system (e.g., image quality assessment system 102 of FIG. 1) selecting a point corresponding to the slice in a graphical element displayed to the user showing details with respect to a quality of the medical image. For example, display panel 1000 may be displayed as a result of the user selecting a point (e.g., point 930) of interactive graph 900 of FIG. 9. Display panel 1000 may include one or more control elements, such as control elements 1004 and 1006, which may be used to change in appearance of 2-D slice 1002. For example, the user may select control element 1004 to view centering marks of 2-D slice 1002, which may be superimposed upon 2-D slice 1002. The centering marks may indicate a uniformity of 2-D slice 1002. If a scanned subject of the medical image is not centered properly, a noise profile of the medical image may be affected, and the quality of the medical image may be deteriorated. Similarly, the user may select control element 1006 to view a noise map of 2-D slice 1002, which may be superimposed upon 2-D slice 1002. In this way, the user may view 2-D slice 1002 with or without the centering marks and the noise map. Display panel 1000 may include directional arrow elements 1008 and 1010, which may be used to advance through a plurality of slices of the medical image. For example, the user may select directional arrow element 1010 to view a slice adjacent to 2-D slice 1002 in a first direction of the medical image, and the user may select directional arrow element 1008 to view a slice adjacent to 2-D slice 1002 and a second, opposing direction of the medical image.

Thus, when the user views an image score of a medical image on a display device of the image quality assessment system, the low-level metric data used to generate the image score may be viewed in various selectable abbreviated representations via summary display panels that display select low-level metric data, without the user having to view comprehensive low-level metric data, which may entail the user having to navigate or scroll through a large number of results, many of which that are not of interest to the user. Rather, the low-level metrics are advantageously displayed in a manner in which the user may view summarized image quality data at various levels of detail, where the user may select elements of data in a first summary display panel to view additional image quality data in a second summary display panel, select elements of data in the second summary display panel to view additional image quality data in a third summary display panel, and so on. When the user selects an element of data to display a summary display panel, the summarized image quality data may be displayed quickly and responsively, as the image quality score generator does not recalculate the low-level metrics or the image quality scores in response to user input.

Returning to FIG. 6, at 606, method 600 includes determining whether an aggregation area within a graphical element including the image quality score (e.g., aggregation area 730 of image quality GUI 700) has been adjusted by the user, as described above in reference to FIG. 7. If at 606 it is determined that the aggregation area has not been adjusted, method 600 proceeds to 608. At 608, method 600 includes continuing to display the image quality score and the low-level metrics in the GUI. Alternatively, if at 606 it is determined that the aggregation area has been adjusted, method 600 proceeds to 610.

At 610, method 600 includes updating the image quality score and/or low-level metrics based on the adjusted aggregation area. In other words, as the aggregation area is adjusted by the user, a number of slices over which the low-level metrics are aggregated to produce the image quality score may change. As a result of the change in the number of slices over which the low-level metrics are aggregated to produce the image quality score, the image quality score will be adjusted. As the image quality score is adjusted, a position of needle 724 of FIG. 7 may be adjusted in a corresponding direction. For example, if the aggregation area is enlarged (e.g., by dragging upper horizontal boundary 704 in direction 752, or by dragging lower horizontal boundary 706 in direction 750), the number of slices taken into consideration in the calculation of the image quality score may increase, and needle 724 may be adjusted in direction 752. Alternatively, if the aggregation area is reduced (e.g., by dragging upper horizontal boundary 704 in direction 750, or by dragging lower horizontal boundary 706 in direction 752), the number of slices taken into consideration in the calculation of the image quality score may decrease, and needle 724 may be adjusted in direction 750.

The results of the low-level metrics are adjusted accordingly. As the number of slices used in the calculation of the image quality score changes, the aggregation of the low-level metrics also changes. For example, a first image quality score may be generated based on a first number of slices, where the first number of slices is based on a first aggregation area. The user may subsequently adjust the aggregation area to form a second aggregation area to focus on a smaller area of interest in the image. A second image quality score may be generated based on a second number of slices, where the second number slices is based on the second aggregation area. The first image quality score may be based on a first set of low-level metrics aggregated over the first number of slices, and the second image quality score may be based on a second set of low-level metrics aggregated over the second number of slices. Before adjusting the aggregation area, the user may select one or more menu items of menu 726, and as a result, a first summary of the first set of low-level metrics aggregated over the first number of slices may be displayed in the GUI. After adjusting the aggregation area, the user may select one or more menu items of menu 726, and as a result, a second summary of the second set of low-level metrics aggregated over the second number slices may be displayed at the GUI. Additionally, the summaries may be advantageously displayed while the image quality score generator is in an unlaunched state. In other words, the generation of the image quality scores may occur prior to adjusting the aggregation area, and the calculation of the aggregated image quality scores may occur in response to the adjustment of the aggregation area by the user, without the image quality score generator generating new image quality scores using the ML model. As a result, the summaries and/or an updated image quality score may be displayed in the GUI in a rapid and responsive fashion, which may encourage the user to provide feedback.

At 612, method 600 includes determining whether the image quality score has been adjusted by the user (e.g., by adjusting a position of needle 724 within slider 722). If at 612 it is determined that image quality score has not been adjusted by the user, method 600 proceeds to 614. At 614, method 600 includes continuing to display the image quality score and the low-level metrics on the display device. Alternatively, if at 612 it is determined that the image quality score has been adjusted by the user, method 600 proceeds to 616.

At 616, method 600 includes storing the adjusted image quality in a memory of the image processing device (e.g., memory 106). In some embodiments, additionally or alternatively, the low-level metrics used to generate the adjusted image quality score and displayed in the GUI may be stored in the memory. Method 600 ends.

Thus, systems and methods are disclosed herein for automatically generating an overall image quality score for a medical image, such as a CT image acquired via a CT imaging system, via an image quality assessment system including an image quality score generator. The overall image quality score may be calculated by the image quality score generator based on a plurality of low-level metrics, using an ML model trained on training data including image quality scores and/or low-level metrics as ground truth data. The overall image quality score may be generated by aggregating image quality scores calculated for slices of the medical image, and/or aggregating image quality scores calculated for specific anatomical regions and/or ROIs of the medical image. The overall image quality score may be displayed in a GUI of the image processing device, within an interactive graphical element that allows a user to provide image quality feedback on the medical image by adjusting the overall image quality score in the GUI. The feedback (e.g., the adjusted overall image quality score) may be stored in a memory of the image quality assessment system, and used to retrain the ML model to increase an accuracy or performance of the ML model.

Additionally, by adjusting an aggregation area of the medical image in the GUI, the user can select a portion of the medical image (for example, where the portion includes an ROI of the medical image), and view an image quality score for the portion of the medical image. In other words, the user may select a plurality of slides of the image over which image quality scores are aggregated, and view an aggregate image quality score corresponding to the plurality of slides in real time. Further, since the image quality score is adjusted in the GUI based on how the image quality scores of selected slides are aggregated, the image quality score may be adjusted without launching the image quality score generator, where the ML model is not used to generate a new image quality score. Rather, the image quality score may be adjusted while the image quality score generator is in an unlaunched state. By calculating the image quality scores in this manner, a responsiveness of the GUI to the user feedback may be increased, such that the user feedback may be quickly and easily provided and stored in the system.

In other words, the methods disclosed herein for generating, displaying, and adjusting the image quality scores and/or low-level image quality metrics improve the capabilities of the image quality assessment system by reducing an amount of processing that would otherwise be performed when regenerating image quality scores using the ML model. Image quality data is generated in a first step by the image quality score generator using the ML model, and the image quality data is aggregated and displayed in a second step during which the image quality score generator is not running. Thus, if the user wishes to view image quality scores of specific portions of the medical image, the image quality scores can be generated quickly by adjusting an aggregation area of the medical image, with minimal processing. In this way, the GUI improves the way the image quality assessment system stores and retrieves data in memory to reduce resource consumption. A specific manner of displaying image quality information to the user based on a limited set of image data is described, such that the user is not burdened by time-consuming, iterative calculations, or by navigating through pages of image quality data amassed for different portions of the medical image. Because the user is not forced to scroll down or navigate through various layers of data to view image quality scores, a rapid and efficient process for collecting image quality feedback is enabled. As a result, ground truth data for refining the ML model may be collected in larger amounts and in shorter time frames than would be permitted by prior art systems and GUIs. Thus, the disclosed invention improves the efficiency of the image quality GUI specifically, and the image quality assessment system in general.

The technical effect of generating an image quality score for a medical image is that an accuracy of parameter settings for acquiring medical images in scan protocols may be increased. The technical effect of displaying image quality scores and low-level image quality metrics for user-selected portions of a medical image is that image quality data sufficient for training or retraining an ML model to generate the image quality scores may be efficiently collected.

In another representation, an image quality assessment system comprises a display device, the image quality assessment system being configured to display in a graphical user interface (GUI) of the display device a medical image, an image quality score indicating a quality of the medical image, the image quality score generated by an image quality score generator of the image quality assessment system; and a menu listing one or more low-level image quality metrics used to generate the image quality score; and additionally being configured to display in the GUI a low-level image quality metric summary that can be reached directly from the menu; wherein the low-level image quality metric summary displays one or more limited sets of data generated based on the low-level image quality metrics, each of the sets of data in the list being selectable to launch a visualization of the set of data; and wherein in response to a user of the image quality assessment system selecting a low-level image quality metric in the menu, the low-level image quality metric summary is displayed in the GUI while the image quality score generator is in an unlaunched state.

In another representation, an image quality assessment system comprises a display device, the image quality assessment system being configured to display in a graphical user interface (GUI) of the display device a medical image, an image quality score indicating a quality of the medical image, the image quality score generated by an image quality score generator of the image quality assessment system; and a menu listing one or more low-level image quality metrics used to generate the image quality score; and additionally being configured to display in the GUI a preview display panel showing a preview of low-level image quality metric data that can be reached directly from the menu; wherein the preview display panel displays one or more limited sets of data generated based on the low-level image quality metrics, each of the sets of data in the list being selectable to launch a visualization of the set of data; and wherein in response to a user of the image quality assessment system selecting a low-level image quality metric in the menu, the low-level image quality metric summary is displayed in the GUI while the image quality score generator is in an unlaunched state.

The disclosure also provides support for a method for an image quality assessment system, the method comprising: receiving a selection of a medical image from a user of the image quality assessment system, generating an image quality score for the selected medical image, the image quality score generated using a trained machine learning (ML) model, displaying the selected medical image and the image quality score in a graphical user interface (GUI) on a display device of the image quality assessment system, receiving an adjusted image quality score of the medical image from the user via the GUI, and using the adjusted image quality score to retrain the ML model. In a first example of the method, generating the image quality score is performed without processing individual components of the medical image pixel-by-pixel. In a second example of the method, optionally including the first example, the medical image is ingested at the image quality assessment system from a Digital Imaging and Communications in Medicine (DICOM) file. In a third example of the method, optionally including one or both of the first and second examples, ingesting the DICOM file further comprises extracting and aggregating metadata of the DICOM file by level. In a fourth example of the method, optionally including one or more or each of the first through third examples, generating the image quality score for the medical image further comprises automatically detecting one or more anatomical regions in the medical image, and associating one or more slices of the medical image with an anatomical region of the one or more anatomical regions. In a fifth example of the method, optionally including one or more or each of the first through fourth examples, generating the image quality score for the medical image further comprises computing a plurality of low-level metrics to evaluate image quality in the one or more slices of the medical image associated with the anatomical region. In a sixth example of the method, optionally including one or more or each of the first through fifth examples, the low-level metrics used to evaluate the image quality include at least one of: a signal-to-noise ratio (SNR), a total amount of noise, a noise power spectrum (NPS), and a contrast-to-noise ratio (CNR). In a seventh example of the method, optionally including one or more or each of the first through sixth examples, the low-level metrics are inputs into the ML model. In an eighth example of the method, optionally including one or more or each of the first through seventh examples, the image quality score for the selected medical image corresponds to a region of interest (ROI) of the selected medical image, the ROI defined by boundaries superimposed on the selected medical image in the GUI, the boundaries repositionable by the user. In a ninth example of the method, optionally including one or more or each of the first through eighth examples, in response to the user repositioning one or more of the boundaries to define a second portion of the selected medical image: displaying an adjusted image quality score in the GUI, the adjusted image quality score corresponding to the second portion of the medical image.

The disclosure also provides support for a medical imaging system comprising a display device, the medical imaging system being configured to display on the display device a menu listing a plurality of slices of a 3-D medical image viewable on the display device, and additionally being configured to display on the display device an image quality graphical user interface (GUI) that can be reached directly from the menu, wherein the image quality GUI displays, for a set of slices of the plurality of slices, an image quality score generated by an image quality score generator, and a limited list of results of low-level image quality metrics applied to the set of slices to generate the image quality score, each result in the limited list being selectable to launch a display panel with additional information relating to the low-level metrics applied to the set of slices and enable at least the selected result to be seen within the display panel, and wherein the image quality GUI is displayed while the image quality score generator is in an unlaunched state. In a first example of the system, the image quality score is based on aggregating the low-level image quality metrics over a plurality of slices of the 3-D medical image, the plurality of slices defined by an aggregation area indicated on a 2-D reference image of the 3-D medical image, and in response to a user adjusting a size and/or position of the aggregation area via the image quality GUI, the image quality score displayed in the image quality GUI is updated while the image quality score generator is in an unlaunched state. In a second example of the system, optionally including the first example, the user adjusts the size and/or position of the aggregation area by repositioning one or more repositionable boundaries of the aggregation area via the image quality GUI. In a third example of the system, optionally including one or both of the first and second examples, the image quality score is indicated in the image quality GUI via an interactive graphical element including a needle, and the image quality score may be adjusted by the user by adjusting a relative position of the needle within the interactive graphical element, and in response to the user adjusting the image quality score, the adjusted image quality score is stored in a memory of the medical imaging system. In a fourth example of the system, optionally including one or more or each of the first through third examples, the adjusted image quality score is used to retrain an ML model used by the image quality score generator to generate the image quality score.

The disclosure also provides support for a method for an image quality assessment system, comprising: receiving a selection of a medical image from a user of the image quality assessment system, displaying the selected medical image in a graphical user interface (GUI) of the image quality assessment system, displaying user-selectable boundaries defining a first portion of the selected medical image, using a machine learning (ML) model to generate a first image quality score indicating an estimated quality of the first portion of the medical image, the ML model taking as input a plurality of low level image quality metrics applied to slices of the selected medical image included in the first portion of the selected medical image, displaying the first image quality score in the GUI, in response to the user adjusting the first image quality score via the GUI, storing the adjusted first image quality score in a memory of the image quality assessment system. In a first example of the method, the first portion of the selected medical image includes the entire selected medical image. In a second example of the method, optionally including the first example, the method further comprises: in response to the user adjusting one or more of the user-selectable boundaries to define a second portion of the medical image, the second portion different from the first portion, displaying a second image quality score in the GUI, the second image quality score indicating an estimated quality of the second portion of the medical image, the second image quality score different from the first image quality score. In a third example of the method, optionally including one or both of the first and second examples, the method further comprises: in response to the user adjusting the second image quality score in the GUI, storing the adjusted second image quality score in the memory of the image quality assessment system. In a fourth example of the method, optionally including one or more or each of the first through third examples, at least one of the adjusted first image quality score and the adjusted second image quality score are used to further train the ML model.

1. A method for an image quality assessment system, the method comprising:
receiving a selection of a medical image from a user of the image quality assessment system;
generating an image quality score for the selected medical image, the image quality score generated using a trained machine learning (ML) model;
displaying the selected medical image and the image quality score in a graphical user interface (GUI) on a display device of the image quality assessment system;
receiving an adjusted image quality score of the medical image from the user via the GUI; and
using the adjusted image quality score to retrain the ML model.

2. The method of claim 1, wherein generating the image quality score is performed without processing individual components of the medical image pixel-by-pixel.

3. The method of claim 1, wherein the medical image is ingested at the image quality assessment system from a Digital Imaging and Communications in Medicine (DICOM) file.

4. The method of claim 3, wherein ingesting the DICOM file further comprises extracting and aggregating metadata of the DICOM file by level.

5. The method of claim 1, wherein generating the image quality score for the medical image further comprises automatically detecting one or more anatomical regions in the medical image, and associating one or more slices of the medical image with an anatomical region of the one or more anatomical regions.

6. The method of claim 5, wherein generating the image quality score for the medical image further comprises computing a plurality of low-level metrics to evaluate image quality in the one or more slices of the medical image associated with the anatomical region.

7. The method of claim 6, wherein the low-level metrics used to evaluate the image quality include at least one of:
a signal-to-noise ratio (SNR);
a total amount of noise;
a noise power spectrum (NPS); and
a contrast-to-noise ratio (CNR).

8. The method of claim 6, wherein the low-level metrics are inputs into the ML model.

9. The method of claim 1, wherein the image quality score for the selected medical image corresponds to a region of interest (ROI) of the selected medical image, the ROI defined by boundaries superimposed on the selected medical image in the GUI, the boundaries repositionable by the user.

10. The method of claim 9, wherein:
in response to the user repositioning one or more of the boundaries to define a second portion of the selected medical image:
displaying an adjusted image quality score in the GUI, the adjusted image quality score corresponding to the second portion of the medical image.

11. A medical system comprising a display device, the medical system being configured to display on the display device a menu listing a plurality of slices of a 3-D medical image viewable on the display device; and additionally being configured to display on the display device an image quality graphical user interface (GUI) that can be reached directly from the menu; wherein the image quality GUI displays, for a set of slices of the plurality of slices, an image quality score generated by an image quality score generator, and a limited list of results of low-level image quality metrics applied to the set of slices to generate the image quality score, each result in the limited list being selectable to launch a display panel with additional information relating to the low-level metrics applied to the set of slices and enable at least the selected result to be seen within the display panel, and wherein the image quality GUI is displayed while the image quality score generator is in an unlaunched state.

12. The medical imaging system of claim 11, wherein the image quality score is based on aggregating the low-level image quality metrics over a plurality of slices of the 3-D medical image, the plurality of slices defined by an aggregation area indicated on a 2-D reference image of the 3-D medical image; and
in response to a user adjusting a size and/or position of the aggregation area via the image quality GUI, the image quality score displayed in the image quality GUI is updated while the image quality score generator is in an unlaunched state.

13. The medical imaging system of claim 12, wherein the user adjusts the size and/or position of the aggregation area by repositioning one or more repositionable boundaries of the aggregation area via the image quality GUI.

14. The medical imaging system of claim 11, wherein the image quality score is indicated in the image quality GUI via an interactive graphical element including a needle, and the image quality score may be adjusted by the user by adjusting a relative position of the needle within the interactive graphical element; and
in response to the user adjusting the image quality score, the adjusted image quality score is stored in a memory of the medical imaging system.

15. The medical imaging system of claim 14, wherein the adjusted image quality score is used to retrain an ML model used by the image quality score generator to generate the image quality score.

16. A method for an image quality assessment system, comprising:
receiving a selection of a medical image from a user of the image quality assessment system;
displaying the selected medical image in a graphical user interface (GUI) of the image quality assessment system;
displaying user-selectable boundaries defining a first portion of the selected medical image;
using a machine learning (ML) model to generate a first image quality score indicating an estimated quality of the first portion of the medical image, the ML model taking as input a plurality of low level image quality metrics applied to slices of the selected medical image included in the first portion of the selected medical image;
displaying the first image quality score in the GUI;
in response to the user adjusting the first image quality score via the GUI, storing the adjusted first image quality score in a memory of the image quality assessment system.

17. The method of claim 16, wherein the first portion of the selected medical image includes the entire selected medical image.

18. The method of claim 16, further comprising:
in response to the user adjusting one or more of the user-selectable boundaries to define a second portion of the medical image, the second portion different from the first portion, displaying a second image quality score in the GUI, the second image quality score indicating an estimated quality of the second portion of the medical image, the second image quality score different from the first image quality score.

19. The method of claim 18, further comprising:
in response to the user adjusting the second image quality score in the GUI, storing the adjusted second image quality score in the memory of the image quality assessment system.

20. The method of claim 19, wherein at least one of the adjusted first image quality score and the adjusted second image quality score are used to further train the ML model.
